SEQUENCE LISTING (4 sequences)

SEQ ID NO: 1 - protein, 15 aa, synthetic construct
  REGION 1..15: note = P10Sol
KKKWKLSGVERAKKK

SEQ ID NO: 2 - protein, 30 aa, synthetic construct
  REGION 1..30: note = P10 30mer
DAAWGGGLLMSRKHKWKLSGVERANSVTWN

SEQ ID NO: 3 - protein, 9 aa, Mus musculus
  SITE 1: note = Acetylation
ASQKRPSQR

SEQ ID NO: 4 - protein, 9 aa, Mus musculus
  SITE 1: note = Acetylation
ASQYRPSQR

(19) United States
(12) Patent Application Publication
(10) Pub. No.: US 2026/0338765 A1
(43) Pub. Date: Oct. 29, 2026

(54) ACTIVATION INDUCED MARKER ASSAY

(71) Applicant: University of Birmingham, Birmingham (GB)

(72) Inventors: David Cameron Wraith, Birmingham (GB); Naomi Richardson, Birmingham (GB); Tien Haeng Ng, Birmingham (GB); Michael John Price, Birmingham (GB)

(21) Appl. No.: 19/099,044

(22) PCT Filed: Jul. 27, 2023
(86) PCT No.: PCT/GB2023/051991

§ 371 Date: Jan. 27, 2025

(30) Foreign Application Priority Data
Jul. 27, 2022 (GB) 2210969.8

(51) Int. Cl. G01N 33/50; C12N 5/0783; G01N 33/58

(52) U.S. Cl. CPC G01N 33/505; C12N 5/0636; G01N 33/582; G01N 2333/70582; G01N 2333/7155

(57) ABSTRACT

The present disclosure is based in part on studies that identify upregulation of markers in response to stimulation by an antigen (also referred to as activation induced markers) to detect antigen-specific immune cells and uses thereof. The disclosure provides methods for identifying antigen-specific T cells produced in response to stimulation by an antigen.

### FIELD

The present disclosure relates to activation induced markers and uses thereof.

### BACKGROUND

Antigen-specific T cells are involved in a range of immune responses, such as towards foreign antigens (e.g., infection), vaccination and self-antigens. Following antigen-induced stimulation, T cells undergo dynamic functional and phenotypic changes, including upregulation of surface expression of activation-associated molecules. Upregulation of cell surface molecules enables identification and isolation of antigen-specific T cells, such as through antibody binding of upregulated surface molecules and subsequent enrichment using various techniques. The ability to detect and isolate antigen-specific T cells is critical for immunological research but accurately identifying antigen-specific T cells is challenging due to their heterogeneity and rarity. Traditional assays for measurement of antigen-specific T cell responses, such as 3H-thymidine incorporation or enzyme-linked immunospot (ELISpot), have readouts, which lack two critical pieces of information; (i) identification of which cell types are responding, and (ii) the phenotype of those responding cells.

More recently, flow cytometry based assays, which measure expression of activation induced markers (AIM), have been developed (Eugster et al., 2013; Dan et al., 2016). When combined with fluorescent cell division tracking dyes, AIM assays seek to identify antigen-responsive CD4+ or CD8+ T cells by proliferation and activation marker expression. Many groups have used variations of the AIM assay and typically utilise combinations of CD25, CD69, CD134 (OX40), CD137 (4-1BB) and CD154 (CD40L) to identify recently activated antigen-responsive T cells, with most involving short-term cultures to a foreign antigen (Eugster et al., 2013; Dan et al., 2016; Havenar-Doughton et al., 2016; Reiss et al., 2017; Bowyer et al., 2018; Jiang et al., 2019; Braun et al., 2020). However, there is a need in the art to develop assays that are capable of culturing T cells for >24 h and/or assays designed to investigate responses to autoimmune self-antigen(s).

It is amongst the objectives of the present disclosure to establish novel activation induced markers through which antigen-specific T cells can be identified, which could mitigate or obviate one or more of the aforementioned disadvantages of existing assays.

### SUMMARY

The present disclosure is based in part on studies that identify upregulation of markers in response to stimulation by an antigen (also referred to as activation induced markers) to detect antigen-specific immune cells, and uses thereof.

In a first aspect, there is provided a method for identifying antigen-specific T cells produced in response to stimulation by an antigen, wherein the method comprises detection of antigen-specific T cell surface expression of CD71, following stimulation by the antigen. In one embodiment, the method further comprises the detection of antigen-specific T cell surface expression of CD25, following stimulation by the antigen.

In a normal adaptive immune response, T cells recognise internal epitopes of an antigen. Antigen presenting cells (APCs) internalise antigens and degrade them into short fragments (antigen processing). An antigen may bind a major histocompatibility complex (MHC) class I or II molecule inside the cell for presentation at the cell surface. The antigen presented on an MHC molecule may be recognised by a T cell, in which case the molecule is a T cell epitope.

In order to activate a T cell, an antigen must associate with a professional APC (e.g., dendritic cells, macrophages, B cells) capable of delivering two signals to T cells. The first signal is delivered by the MHC-peptide complex on the cell surface of the APC and is received by the T cell via the T cell receptor (TCR). The second signal is delivered by costimulatory molecules on the surface of the APC, such as CD80 and CD86, and received by CD28 on the surface of the T cell. T cells that recognise and respond to stimulation by an antigen presented on an MHC molecule in the presence of the abovementioned signals from an APC are referred to as antigen-specific T cells. In a preferred embodiment, the antigen-specific T cells as described herein are CD4+ and/or CD8+.

Antigen refers to a macromolecule or a portion thereof, typically a protein or peptide (with or without polysaccharides), capable of eliciting an immune response. The skilled person in the art would recognise that any molecule, including naturally occurring macromolecules or synthetic variant(s) thereof, may serve as an antigen. The antigen used to produce antigen-specific T cells may be a natural epitope sequence of a protein, or may be a modified variant thereof (including synthetic variants) provided that the modified variant retains its ability to bind an MHC molecule similar to a natural T cell epitope sequence.

In one embodiment, the method for identifying antigen-specific T cells produced in response to stimulation by an antigen, as described herein, comprises an antigen that may be a self-antigen or a foreign antigen. In one embodiment, the antigen may be derived from cellular proteins, peptides, enzyme complexes, ribonucleoprotein complexes, nucleic acids and/or post-translationally modified proteins.

In one embodiment, the method for identifying antigen-specific T cells produced in response to stimulation by an antigen as described herein comprises an antigen that is a foreign antigen. Foreign antigens may include any substance originating from outside the body, such as a component of or a substance produced by viruses or microorganisms. Other examples of foreign antigens may include chemicals, toxins and proteins in certain food products, for example.

In a particular embodiment, the method of the present disclosure may be used to identify antigen-specific T cells wherein the T cells are produced in response to a foreign antigen or a neoantigen, such as to test the efficacy of vaccines. The foreign antigen may comprise an active ingredient of a vaccine against microorganism(s), such as a component (e.g. protein or glycoprotein) derived from a protein of a pathogen, such as a virus or bacterium. A neoantigen typically refers to a tumour neoantigen, which is an antigen that has at least one alteration that makes it distinct from the corresponding wild-type parental antigen. For instance, in some cancers tumour-specific neoantigens may be generated by mutation(s) or post-translational modification(s) in tumour cells, and these neoantigens are predominantly expressed by these tumour cells. In some embodiments, the antigen may include a peptide sequence, polypeptide sequence or a nucleic sequence. The nucleic acid sequence may encode the peptide or polypeptide sequence that serve as an antigen.

Alternatively, the antigen as described herein may be a self-antigen (e.g. antigen derived from a protein or complex of proteins), such as an antigen capable of inducing an autoimmune disease. Self-antigen or auto-antigen refers to a human or animal antigen present in the body, which elicits an immune response within the same human or animal body. Self-antigens may comprise, but are not limited to, cellular proteins, phosphoproteins, cellular surface proteins, cellular lipids, nucleic acids, glycoproteins, for example.

Self-antigens associated with various autoimmune diseases are known in the art. For example, self-antigens associated with type 1 diabetes include glutamate decarboxylase, insulin and proinsulin; self-antigens associated with multiple sclerosis include myelin basic protein (MBP), myelin oligodendrocytic glycoprotein and proteolipid protein; self-antigen associated with rheumatoid arthritis include citrullinated protein and collagen; self-antigens associated with systemic lupus erythematosus include double-stranded DNA and nucleosomal histones and ribonucleoproteins. In one embodiment, the method as described herein may be directed towards identifying antigen-specific T cells involved in type 1 diabetes, wherein the self-antigen may be derived from a pancreatic islet protein. In certain embodiments, the antigen may be derived from glutamate decarboxylase 65, tyrosine phosphatase-related islet antigen 2 and/or pro-insulin.

In an alternative embodiment, the method described herein may be used to identify antigen-specific T cells that are tolerant towards self-antigens and thereby have the potential to elicit immune tolerance. Immune tolerance refers to a range of host processes that prevent potentially harmful immune responses and results in a state of unresponsiveness of the immune system to substances or tissue(s) that have the capacity to elicit an immune response. Immune tolerance is a highly regulated process that enables the discrimination of self from non-self, suppression of allergic responses and prevention of reactive immune responses towards foetal antigens by the maternal immune system.

Subsequent to activation by an antigen, such as those described above, T cells proliferate and undergo phenotypic and functional changes to carry out effector functions, which typically involves altering expression of specific markers. Activation induced markers refer to molecules that are expressed or upregulated by T cells in response to stimulation by an antigen. Typically, activation induced markers comprise cell surface molecules, such as receptors or components thereof, that are expressed or upregulated subsequent to T cell activation in response to stimulation by an antigen.

Surprisingly, the inventors have identified that detection of T cell surface expression of CD71, and optionally CD25, improves the ability to identify antigen-specific T cells produced in response to stimulation by an antigen. In addition to detection of CD71, and optionally CD25, it is envisaged that it would be possible to combine other components upregulated by activated T cells in response to stimulation by an antigen to enhance detection of such T cells. In one embodiment, the detection of antigen-specific T cells further comprises detection of antigen-specific T cell surface expression of CD69, CD40L, OX40, 4-1BB, PD-L1, CD107a, or any combination thereof. Alternatively, or in addition to the abovementioned markers, in one embodiment, the detection of antigen-specific T cells further comprises detection of antigen-specific T cell surface expression of CD3 and ICOS, or any combination thereof.

In one embodiment, the method may comprise culturing of T cells for at least one, two, three, four or five days. In one embodiment, the method comprises culturing of T cells for at least three days. In a preferred embodiment, the method comprises culturing of T cells for at least four days. Importantly, the present disclosure provides a method whereby cells can be cultured over the course of several days. The method disclosed herein surprisingly enables the selective expansion of antigen-specific cells which improves signal to noise ratio. In one embodiment, the method comprises culturing T cells with a self-antigen or a foreign antigen. In one embodiment, the method comprises culturing T cells with a self-antigen. In some embodiments, the culturing of T cells may be prior to the detection of T cell surface expression of the abovementioned activation induced markers. In alternative embodiments, the culturing of T cells may be subsequent to the detection and isolation of T cells with surface expression of the abovementioned markers.

Detection of antigen-specific T cell surface expression of the markers described herein may comprise detection of a colorimetric, fluorescent or luminescent signal, for example. The ordinary skilled person will recognise that various detection methods known in the art may be used to detect surface expression of antigen-specific T cells of one or more activation induced markers as described herein. In a preferred embodiment, the antigen-specific T cells are identified through detection of a fluorescent signal. In one embodiment, the antigen-specific T cells are detected by a fluorophore conjugated to an antibody, aptamer or the like capable of binding an antigen-specific T cell surface expression marker as described herein. In an alternative embodiment, the antigen-specific T cells are detected by a colorimetric or luminescent moiety conjugated to an antibody, aptamer or the like capable of binding an antigen-specific T cell surface expression marker. Subsequently, the identified antigen-specific T cells may be optionally isolated using various methods such as flow cytometry based methods (e.g. a fluorescence-activated cell sorting (FACS) technique, known in the art and described herein).

Another feature associated with T cell activation in response to an antigen is the induction of cell proliferation. In one embodiment, the identification of antigen-specific T cells may further comprise labelling of T cells with a proliferation marker. In one embodiment, the proliferation marker comprises a colorimetric, fluorescent or luminescent molecule. In one embodiment, the proliferation marker used to label T cells comprises a cell permeant molecule. In one embodiment, the cell permeant proliferation marker may be a fluorescent molecule. In an alternative embodiment, the cell permeant proliferation marker may comprise a non-fluorescent precursor compound that emits a fluorescent signal once inside the cytoplasm or an intracellular compartment of a cell. For instance, the non-fluorescent precursor compound may comprise one or more acetate substituents capable of diffusing across the cell plasma membrane, where the acetate substituent(s) are cleaved by non-specific esterase(s) in the cytoplasm. The cleavage promotes trapping of the charged fluorescent product and random protein labelling via covalent bond formation between free amino substituents and the dye's succinmidyl esters. In a preferred embodiment, the cell permeant proliferation molecule comprises a non-toxic stain, such as carboxyfluorescein succinimidyl ester, CellTrace Violet or the like.

In a preferred embodiment, the method comprises identifying antigen-specific T cells produced in response to stimulation by an antigen, wherein said method comprises detection of antigen-specific T cell surface expression of CD71 and CD25, and labelling of the T cells with a proliferation marker comprising a cell permeant molecule. In one embodiment, the proliferation marker may be selected from CellTrace Far Red, CellTrace Yellow, CellTrace Blue, Cell Proliferation Dye eFluor670, Cell Proliferation Dye eFluor450 LuminiCell Tracker 670, LuminiCell Tracker 540, Cyto Track Blue, Cyto Track Green, Cyto Track Yellow, Cyto Track Red, or a combination thereof. In one embodiment, the proliferation marker may be CellTrace Violet. In one embodiment, the proliferation marker may be carboxyfluorescein succinimidyl ester. The present inventors have unexpectedly identified that the sensitivity of the method of identifying antigen-specific T cells may be improved by detecting cell-surface expression of CD71 and CD25 in combination with tracing cell proliferation through the use of a proliferation marker.

In another aspect of the present disclosure, there is provided an isolated CD71+ and CD25+ antigen-specific T cell wherein the T cell is CD4+ and/or CD8+. Further, the isolated antigen-specific T cell may be CD69+, CD40L+, OX40+, 4-1BB+, PD-L1+, CD107a+, or any combination thereof. Optionally, the isolated antigen-specific T cell may further be CD3+, ICOS+, or any combination thereof. In a preferred embodiment, the isolated antigen-specific T cell is proliferative. In some embodiments, the isolated antigen-specific T cell may be labelled with a proliferation marker, wherein the marker comprises a cell permeable molecule. In some embodiments, there is provided isolated CD71+ and CD25+ antigen-specific regulatory T cells for use in the treatment of autoimmune disease. In some embodiments, there is provided CD71+ and CD25+ neoantigen specific T cells for use in the treatment of autoimmune disease or cancer.

The present disclosure is also directed to the use of T cell surface expression of CD71 in the identification of antigen-specific T cells produced in response to stimulation by an antigen. In one embodiment, the use of T cell surface expression of CD71 to identify antigen-specific T cells may additionally comprise T cell surface expression of CD25. In one embodiment, the antigen-specific T cells may be produced in response to a self-antigen or a foreign antigen. In one embodiment, the antigen-specific T cells may be produced in response to a self-antigen. In one embodiment, the use of antigen-specific T cells produced in response to an antigen stimulation comprises any one or more of the antigens as described herein.

The present disclosure may also be directed to the use of antigen-specific T cells in the method as described herein, wherein the antigen-specific T cells may be used for functional analysis. In one embodiment, the functional analysis may include, but are not limited to, transcriptional analysis, proteomic analysis or cytokine analysis using methods known in the art. In one embodiment, the antigen-specific T cells as described herein may be used for testing efficacy of vaccines, immunotherapies or drugs. In an alternative embodiment, the use of the antigen-specific T cells in the method as described herein may be for T cell cloning, T cell receptor cloning and/or sequencing. In one embodiment, the use of the antigen-specific T cells in the method as described herein may be for single-cell TCR cloning and/or sequencing, such as for determination of TCR sequences. The identified TCR sequences may be used to transfect into TCR knockout reporter cell lines to test antigen reactivity.

### DETAILED DESCRIPTION

The present disclosure is further described by way of example and with reference to the figures, which show:

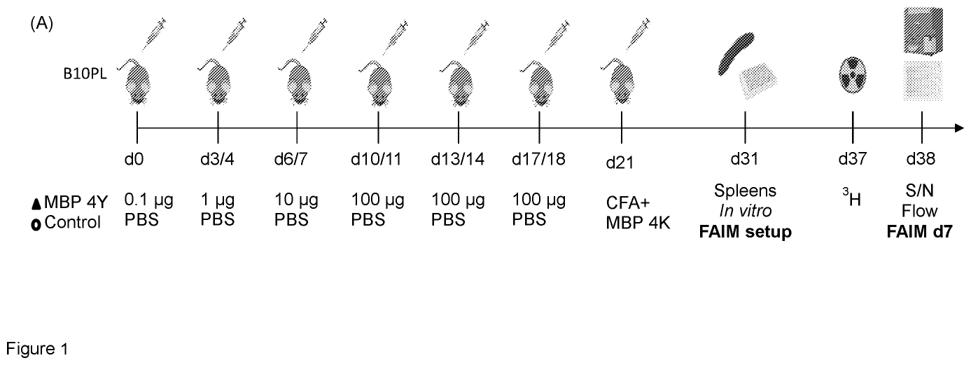

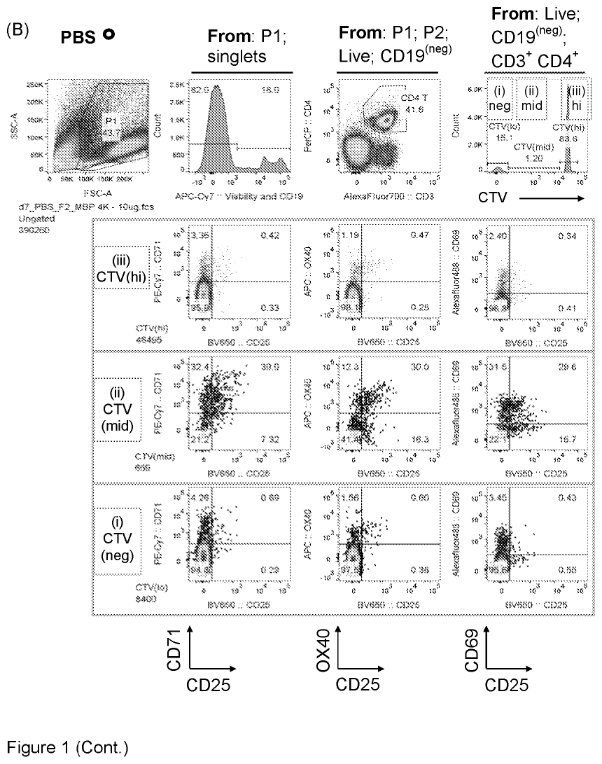

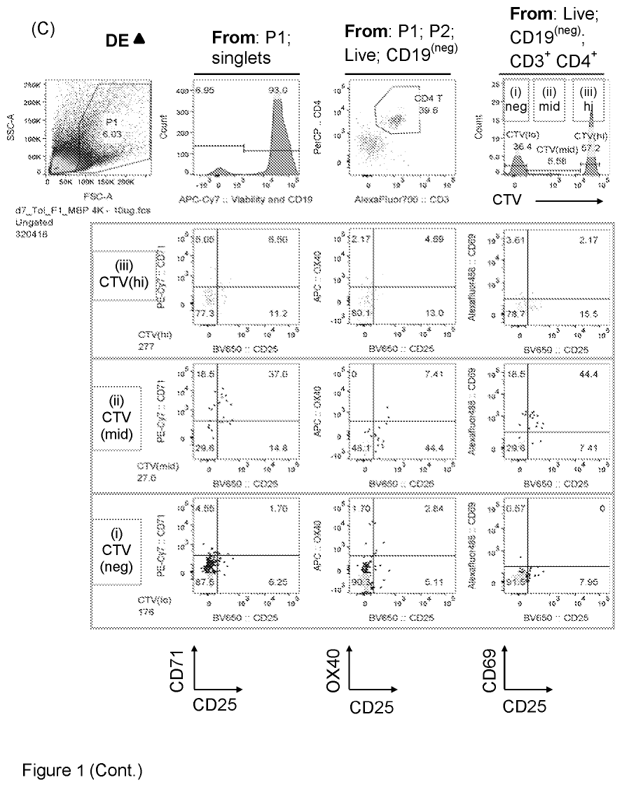

FIG. 1. Use of the Fluorescent dye dilution Activation Induced Marker (FAIM) assay to measure T cell tolerance induction by a known tolerogenic soluble solution peptide. (A) Schematic describes schedule of PBS control or dose escalation of known tolerogenic peptide from myelin basic protein (MBP) Ac1-9[4Y] (MBP 4Y) in B10.PL mice, challenge, and Fluorescent dye dilution and Activation Induced Marker (FAIM) assay readouts to test whether the FAIM assay can distinguish induction of tolerance. Created with BioRender.com. Challenge; both MBP 4Y dose escalation and PBS control groups challenged with 100 μg MBP Ac1-9[4K] (MBP 4K) in Complete Freund's Adjuvant (CFA), spleens and in vitro; splenocytes isolated for CD4 selection, CTV labelling, recombination with unlabelled splenocytes and in vitro re-stimulation and culture, ³H; aliquot taken for paired ³H-thymidine proliferation assay, S/N; culture supernatants analysed for cytokine production, flow; flow cytometric analysis. (B-C) Gating schematics to demonstrate how proliferating (CTV^{(mid)}) CD4 T-cells were analysed for marker expression. Positive expression gates for markers CD25, CD71, OX40 and CD69 were set using non-responding CTV labelled CD4 T-cells (CTV^{(hi)}) and applied to proliferated CTV^{(mid)} CD4 T-cells. Cells from PBS treated mouse re-stimulated in vitro with 10 μg/mL MBP 4K (B), and cells from MBP 4K dose escalation treated mouse re-stimulated in vitro with 10 μg/mL MBP 4K (C).

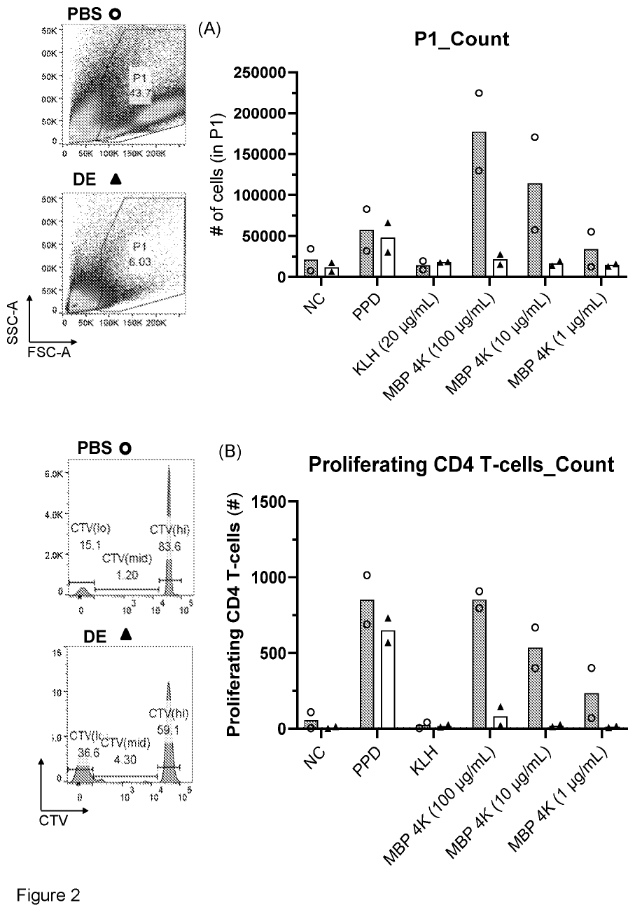

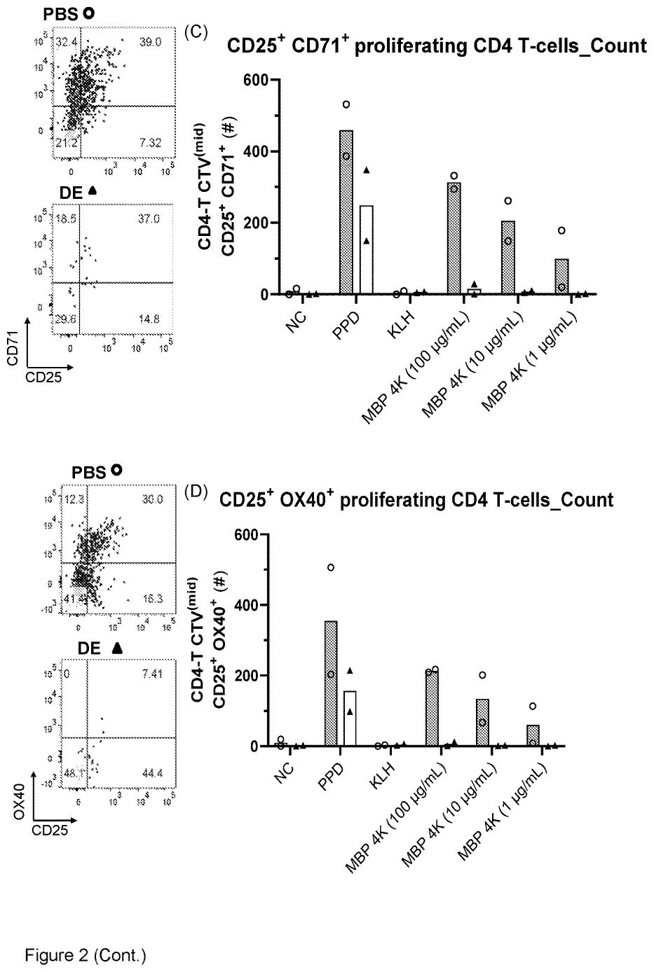

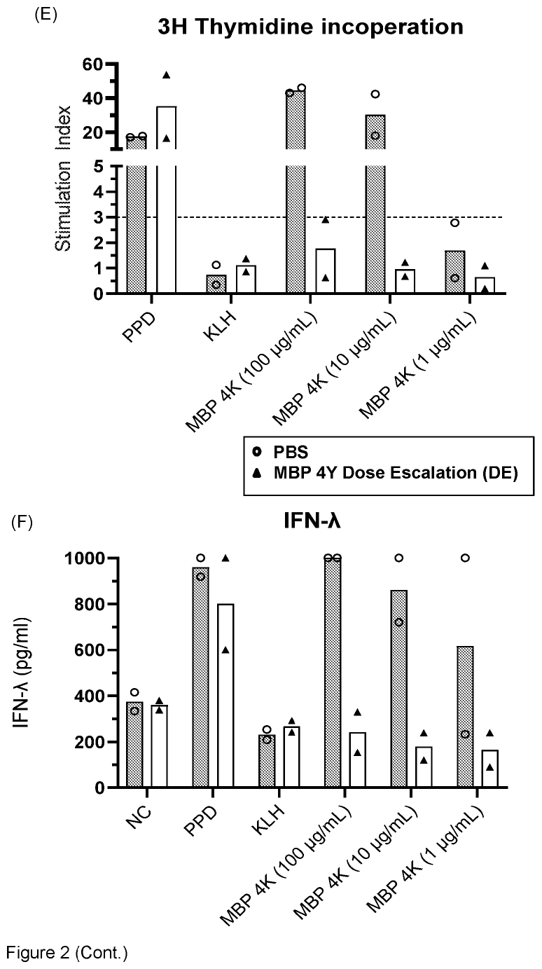

FIG. 2. T-cell tolerance induced by MBP Ac1-9[4Y] is shown by the FAIM assay. Tolerance induction assessed via flow cytometric parameters; lymphocytes and blasting cell count (A), proliferating CTV^{(mid)} CD4 T-cell count (B), CD25⁺ CD71⁺ proliferating CD4 T-cell count (C), CD25⁺ OX40⁺ proliferating CD4 T-cell count (D), paired ³H-thymidine incorporation data (E) and IFN-γ secretion into culture supernatant (F). For ³H-thymidine data, threshold stimulation index (SI) of 3 indicated by dotted black line. PBS treated mice n=2, MBP 4Y dose escalation treated mice n=2, in one independent experiment.

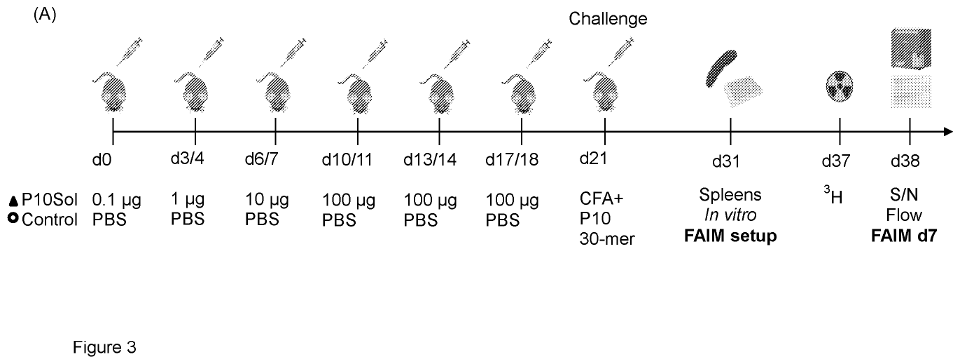

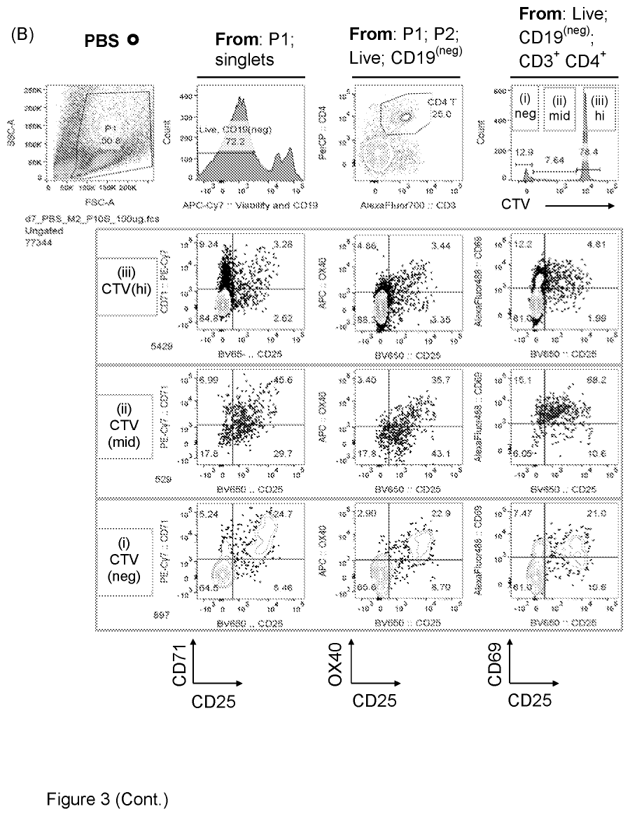

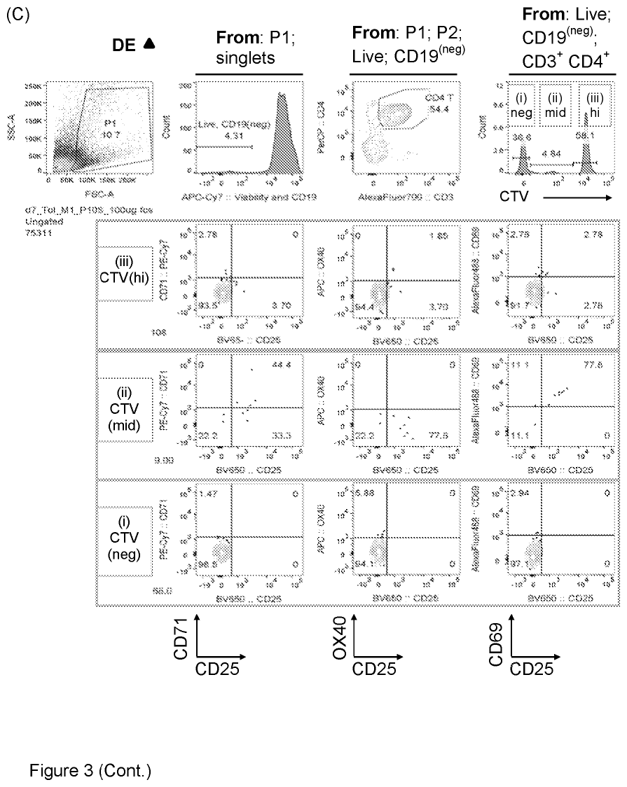

FIG. 3. Use of FAIM assay for assessment of whether GAD65 P10Sol peptide of unknown tolerogenic potential can induce T-cell tolerance. (A) Schematic to show mouse treatment schedule, challenge, FAIM assay and readouts to test whether GAD65 P10Sol can induce tolerance in DR4 transgenic mice. Created with BioRender.com. Challenge; both GAD65 P10Sol dose escalation and PBS control groups were challenged with 100 μg GAD65 P10 30-mer in Complete Freund's Adjuvant (CFA), spleens and in vitro; splenocytes isolated for CD4 selection, CTV labelling, recombination with unlabelled splenocytes and in vitro re-stimulation and culture, ³H; aliquot taken for paired ³H-thymidine proliferation assay, S/N; culture supernatants analysed for cytokine production, Flow; flow cytometric analysis. (B-C) Gating schematics to demonstrate how proliferating (CTV^{(mid)} CD4 T-cells were analysed for FAIM expression. Positive expression gates for markers CD25, CD71, OX40 and CD69 were set using non-responding CTV labelled CD4 T-cells (CTV^{(hi)}) and applied to proliferated CTV^{(mid)} CD4 T-cells. Cells from PBS treated mouse re-stimulated in vitro with 100 μg/mL GAD65 P10Sol (B), and cells from GAD65 P10Sol dose escalation treated mouse re-stimulated in vitro with 100 μg/mL GAD65 P10Sol (C).

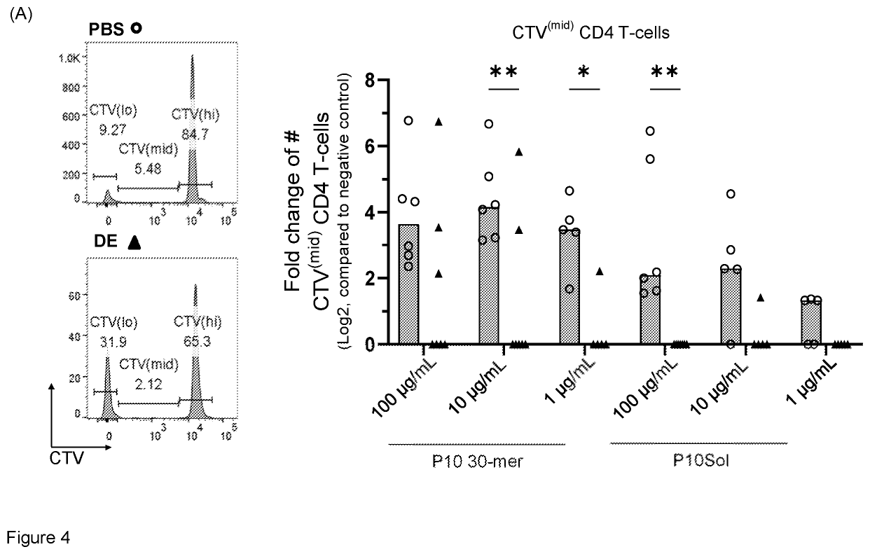

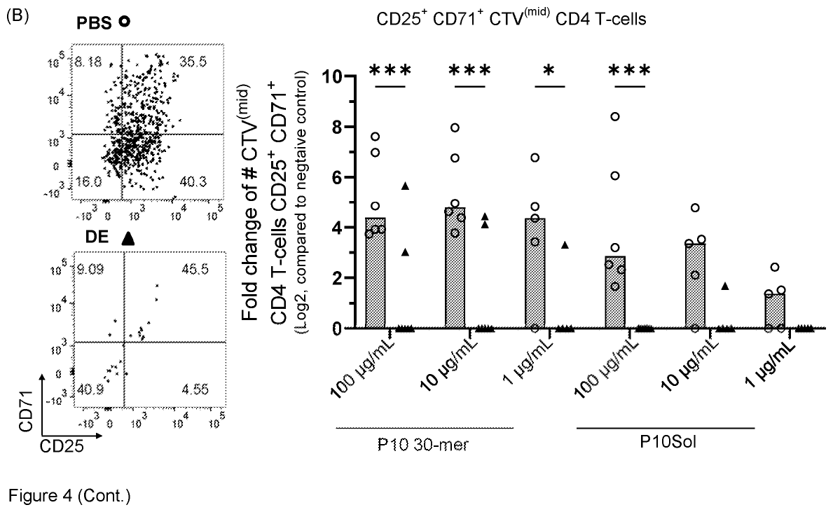

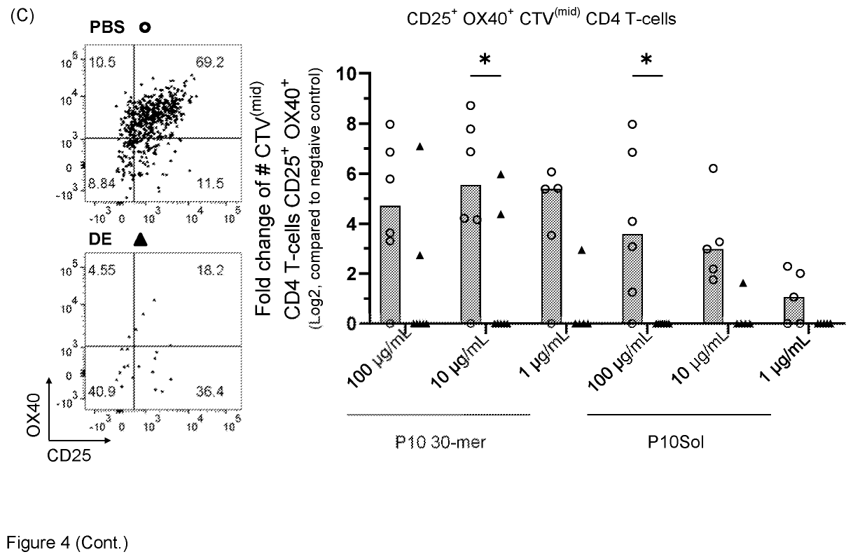

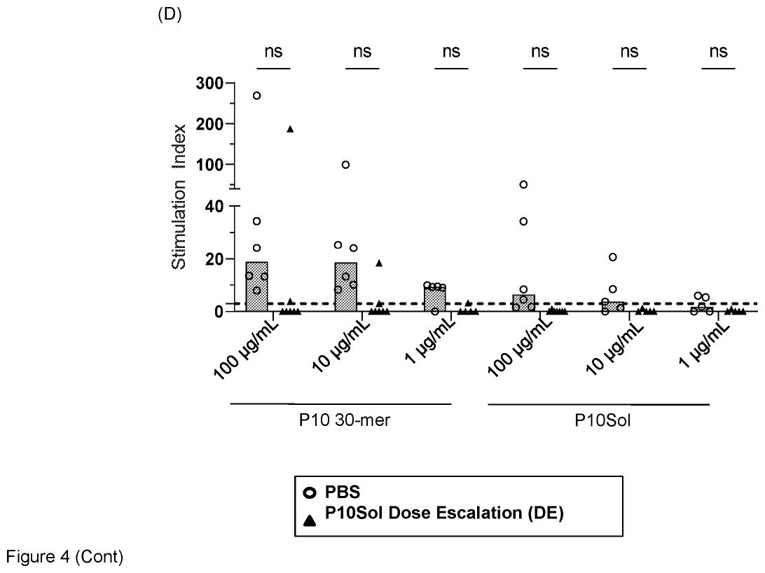

FIG. 4. T-cell tolerance induced by GAD65 P10Sol is demonstrated by the FAIM assay. Tolerance induction assessed via flow cytometric parameters; proliferating CTV^{(mid)} CD4 T-cell count (A), CD25⁺ CD71⁺ proliferating CD4 T-cell count (B), CD25⁺ OX40⁺ proliferating CD4 T-cell count (C) and by paired ³H-thymidine incorporation data (D). For ³H-thymidine data, threshold stimulation index (SI) of 3 indicated by dotted black line. PBS treated mice n=6, P10Sol dose escalation treated mice n=7, from three independent experiments. Šídák's multiple comparison test used in (A-D).

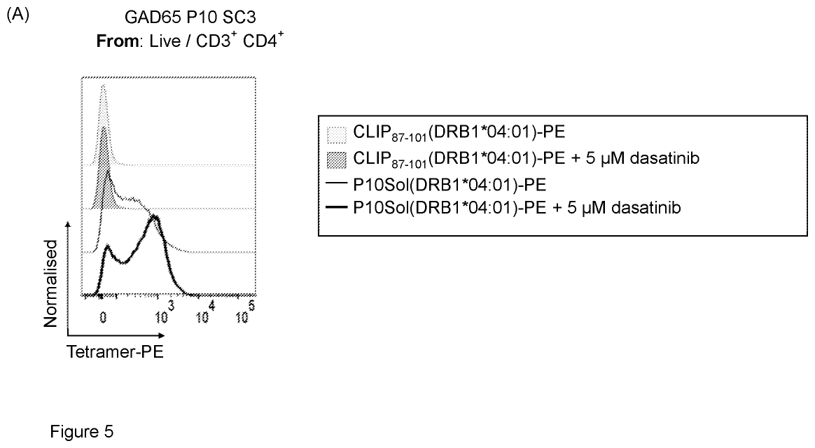

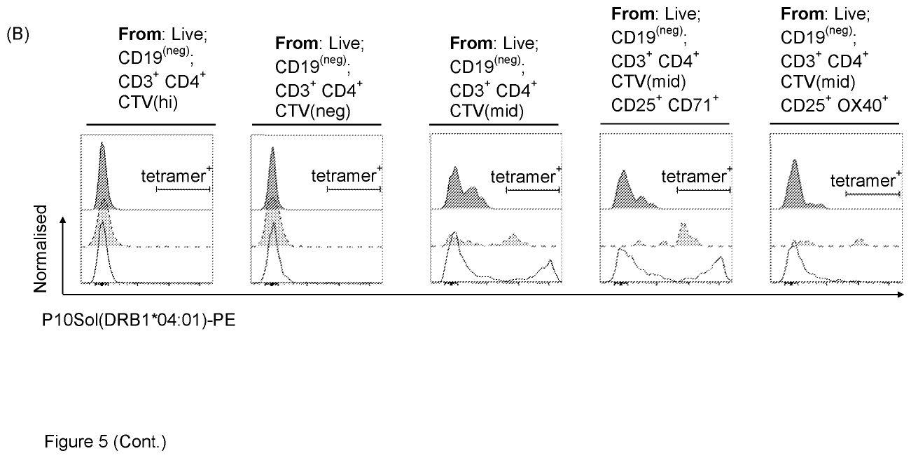

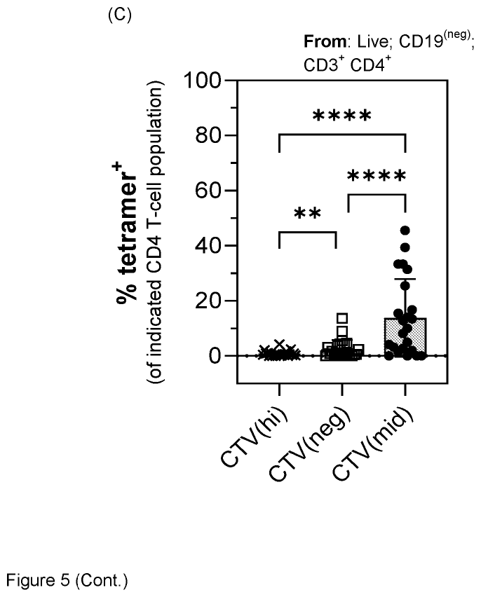

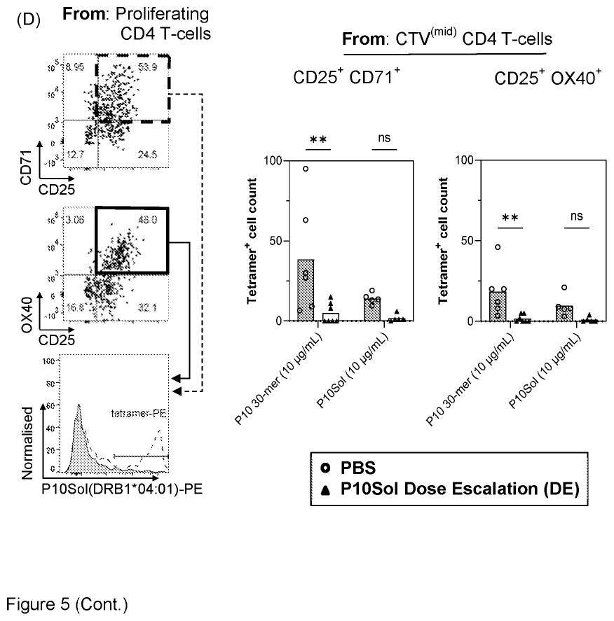

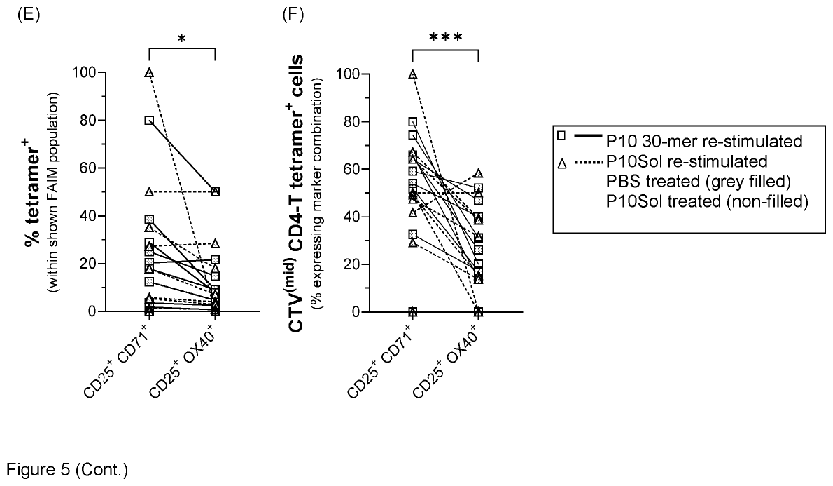

FIG. 5. GAD65 P10Sol MHC class II tetramer staining is enriched in CD71⁺ FAIM⁺ responding CD4 T-cells. (A) Validation of P10Sol (DRB1*04:01)-PE tetramer. GAD65 P10 SC3 hybridoma cells were either pre-incubated at 37 °C for 10 mins in media only or media with 5 μM dasatinib before being stained at 37 °C for 1 h in the dark with either P10Sol (DRB1*04:01)-PE tetramer or control CLIP₈₇₋₁₀₁(DRB1*04:01)-PE tetramer (both at 15 nM final concentration) in a stain mix which contained 5 μM dasatinib if the cells were pre-treated with dasatinib. This was followed by cell-surface anti-CD3, anti-CD4 and viability stain. Tetramer-PE overlays are from live, CD3⁺ CD4⁺ GAD65 P10 SC3 hybridoma cells. (B-F) P10Sol(DRB1*04:01)-PE tetramer staining in the FAIM assay used to test P10Sol tolerance induction described in FIGS. 3 and 4. (B) Example histogram overlays for P10Sol (DRB1*04:01)-PE tetramer staining in the indicated cell population. Fluorescence minus one (FMO) control are cells from a PBS treated mouse re-stimulated with 100 μg/mL GAD65 P10Sol and stained for cell-surface markers excluding the P10Sol (DRB1*04:01)-PE tetramer stain, black shaded trace (top). Cells from a PBS treated mouse re-stimulated in vitro with 10 μg/mL GAD65 P10 30-mer with full staining panel, solid line unfilled trace (bottom). Cells from a GAD65 P10Sol dose escalation treated mouse re-stimulated in vitro with 10 μg/mL GAD65 P10 30-mer with full staining panel, dashed line shaded trace (middle). (C) Tetramer binding within non-responsive (CTV^{(hi)}) CD4 T-cells, proliferating CD4 T-cells (CTV^{(mid)}), and CTV negative CD4 T-cells (CTV^{(neg)}). (D) Example overlay comparison of P10Sol (DRB1*04:01)-PE tetramer staining in CD25⁺ CD71⁺ (dashed line unfilled trace) or CD25⁺ OX40⁺ (solid line shaded trace) FAIM⁺ populations from PBS treated mouse re-stimulated with 10 μg/mL P10 30-mer (D, left), with tetramert absolute numbers in FAIM⁺ cells separated by treatment type (D, right). PBS treated mice, open circles. P10Sol dose escalation treated mice, black filled triangles. Comparison of tetramer positivity rate within CTV^{(mid)} FAIM⁺ population (G) and the frequency of CTV^{(mid)} tetramer+CD4 T-cells which express FAIM combinations (H). PBS treated mice n=6, P10Sol dose escalation treated mice n=7, from three independent experiments. Statistical tests: Tukey's multiple comparisons test (C), paired t-(G) and Wilcoxon matched pairs signed rank (H).

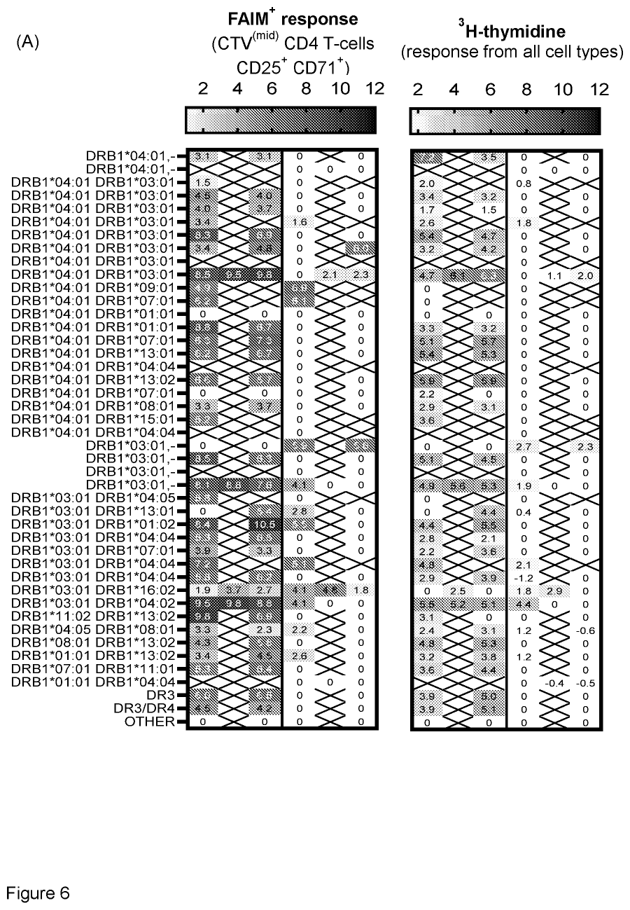

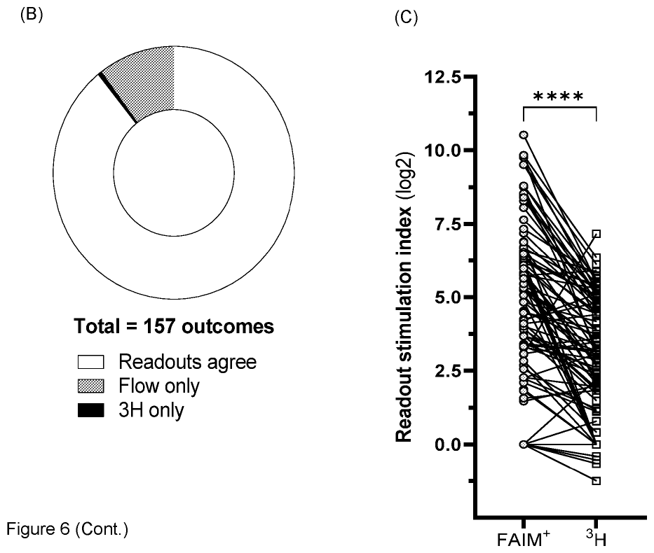

FIG. 6. Self-antigen responses can be measured by the FAIM assay. (A) PBMCs from patients with T1D were screened using the FAIM assay for responsiveness to GAD65 P10 30-mer and GAD65 P10Sol with paired ³H-thymidine samples were acquired for each condition. A positive response for FAIM assay required a proliferating CD25⁺ CD71⁺ CD4 T-cell count >25 and the population SI to be ≥2× the negative control. A positive response for the ³H-thymidine assay required raw counts >1000 and a SI of ≥3× the negative control. White cells with the number zero indicate that this condition failed to induce a response which met the assay readout positive threshold, whereas a cell with a cross indicates that peptide concentration was not tested for that patient. The scale for both heat maps is Log2(stimulation index (SI)). Patient data is ordered by the presence of T1D associated DRB1 sub-alleles where mid-resolution genotype data was available or by HLA-DR serotype where only low-resolution typing was available. DRB1*04:01,—indicates homozygosity for the allele. (B) Agreement analysis for the outcomes of the flow cytometric and 3H-thymidine proliferation readouts of the FAIM assay. (C) Paired analysis of stimulation indexes from FAIM+ and ³H-thymidine readouts, wilcoxon matched-pairs sign rank test.

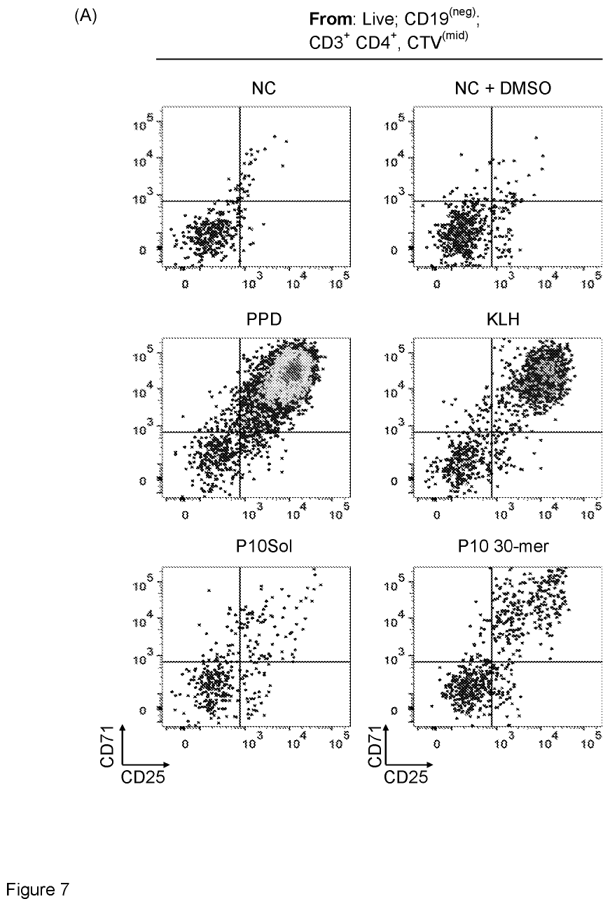

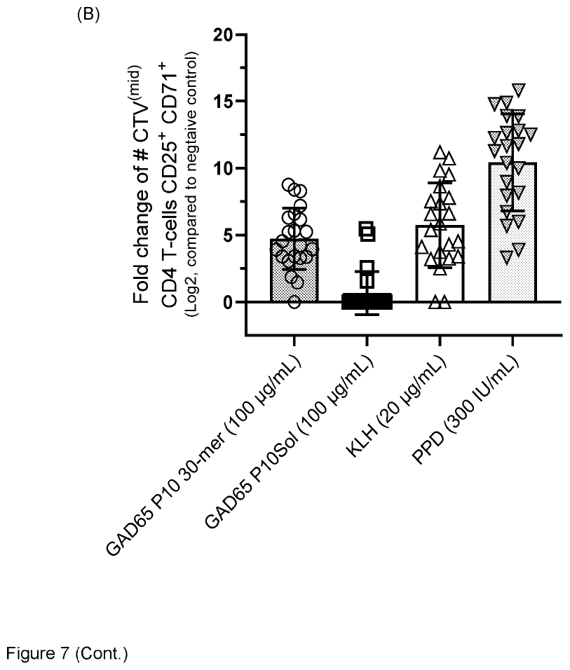

FIG. 7. Foreign antigen responses can be measured by the FAIM assay. (A) Example flow cytometric plots from day 7 of the FAIM assay which stimulated PBMCs from a single T1D patient with the indicated self- or foreign-antigen or control. (B) The magnitude of responses induced in PBMCs from 22 individuals with T1D re-stimulated in vitro with self- or foreign antigens. Data points at zero indicate that stimulant failed to induce a response which met the FAIM⁺ positive response criteria.

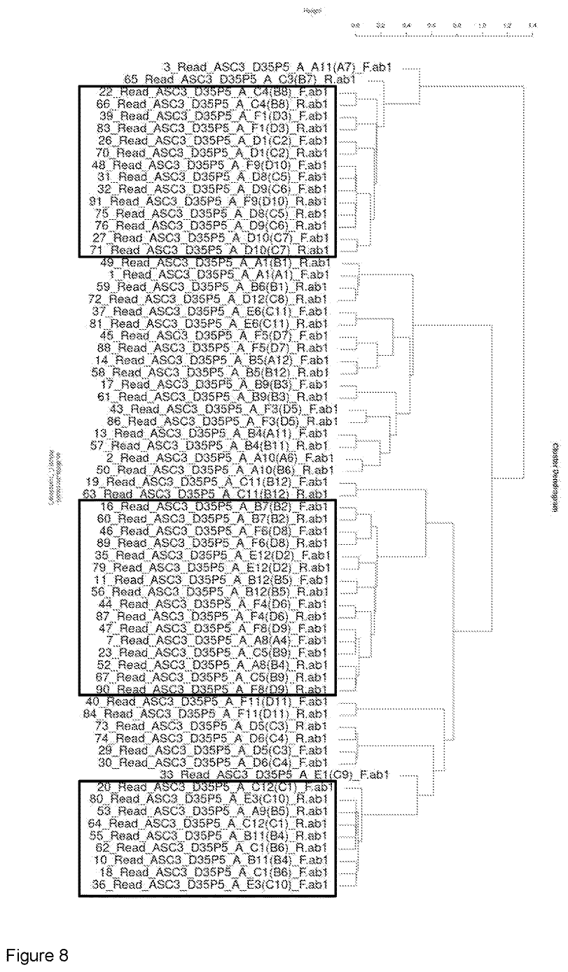

FIG. 8. Identification of clonally expanded antigen-responsive T-cells by the FAIM assay. PBMCs from a healthy donor were stimulated with pro-insulin whole protein in the FAIM assay. On day 7 of culture CD25⁺ CD71⁺ CTV^{mid} (FAIM⁺) CD4 T-cells were single-cell sorted for TCR cloning. TCR α genes were isolated, sequenced and analysed. Dendrogram generated using sangeranalyseR and includes forward (xxx_F.abi) and reverse (xxx_R.abi) sequencing reactions for each TCR α sequence. Thick black boxes surround clusters of CD4 T-cells which possess TCR α sequences with high similarity.

### METHODS

### Mice

B10.PL-H2u H2-T18a/(73NS)SnJ (B10.PL) male and female mice were purchased from The Jackson Laboratory.

HLA-DR4 transgenic mice expressing HLA-DRA*01:01, -DRB1*04:01 and human CD4 previously described (Fugger et al. 1994). B-cells from the peripheral blood of HLA-DR4 mice were phenotyped for human HLA (clone TÜ39) and mouse MHCII (clone M5/114.15.2) by flow cytometry.

Male and female experimental mice, aged between 6-12 weeks, were housed under specific pathogen-free conditions in the Biomedical Services Unit, University of Birmingham. Experiments were performed in accordance with the local ethical review panel and UK Home Office regulations.

### HLA-DR Typing

Genomic DNA was extracted from 1-5×10⁶ PBMCs (Qiagen; 69504). Low-resolution HLA-DR serotype was interpreted from the positive lanes after PCR analysis using the reagents and results tables from the HLA-DR Low typing kit (Olerup; 101.101-12u). Mid resolution HLA-DRB1 genotyping was provided by VH Bio.

### Antigens

T cells were stimulated with an antigen, or a fragment thereof, selected from: foreign antigens—purified protein derivative (PPD; Prionics 7600060) or keyhole limpet haemocyanin (KLH; ThermoFisher Scientific 77600); autoimmune antigens—acetylated native N-terminal murine myelin basic protein (MBP) Ac1-9[4K] (AcASQKRPSQR), MBP Ac1-9[4Y] (AcASQYRPSQR), human pro-insulin protein (Biologics Corporation), human glutamate decarboxylase (GAD65) protein (Biologics Corporation), or GAD65 peptides synthesized by GL Biochem (Shanghai) Ltd or GenScript (Leiden, The Netherlands). Peptides were >90% purity, resuspended from lyophilised powder in either 100% v/v DMSO for 30-mers or PBS for soluble peptides.

### Human/Mouse Fluorescent Dye Dilution Activation Induced Marker (FAIM) Assay

CD4⁺ cells were isolated from fresh T1D patient PBMCs or splenocytes from HLA-DR4 transgenic (Miltenyi Biotec; 130-096-533 or BioLegend; 480033) and labelled with 5 μM CellTrace Violet (CTV) according to manufacturer's instructions (Invitrogen; C34557). For the human FAIM assay, CTV labelled CD4⁺ cells and untouched PBMCs were resuspended in human X-VIVO media (X-VIVO-15 (Lonza; BE02-061Q); 5% v/v human AB serum (Sigma; H4522); 1× penicillin/streptomycin (Gibco; 15140122)), whereas the mouse FAIM assay media lacked human AB serum. CTV labelled CD4⁺ cells and untouched PBMCs/splenocytes were mixed at a ratio of 1:2 and plated at 1.5×10⁶ cells/mL in either a 24-well or 48-well flat bottom plate (1 mL or 0.5 mL cells/well respectively) before the appropriate stimulant or control added. Cells were cultured for 6 days at 37 °C before 20% of cell culture volume was transferred to a 96 round bottomed plate and pulsed with 3H-thymidine for 18-24 h at 37 °C for paired ³H thymidine incorporation data. Criteria for a positive response by ³H-thymidine incorporation were raw counts >1000 and a stimulation index (SI) ≥3, calculated as the fold-change in raw counts of the peptide stimulated condition over the negative control. For the flow cytometric FAIM readout, the remaining cell culture volume was incubated for the full 7 days before staining for cell-surface markers by incubation with antibody mixes for 20 min in the dark at 4 °C. Data acquisition was performed with a BD Fortessa X-20 using DIVA software. Antibodies/stains for mouse FAIM assay; near infra-red fixable viability (Invitrogen; L34975), CD19 APC-Cy7 (BioLegend; 115530), CD4 PerCP-Cy5.5 (BioLegend; 100540), CD3 AlexaFluor700 (BioLegend; 100215), CD25 BV650 (BioLegend; 102037), CD69 FITC (BioLegend; 104506), CD71 PE-Cy7 (BioLegend; 113811) and OX40 APC (BioLegend; 119413). Antibodies/stains for human FAIM assay; yellow fixable viability (Invitrogen; L34967), CD14 APC-Cy7 (BioLegend; 367108), CD16 APC-Cy7 (BioLegend; 302018), CD19 APC-Cy7 (BioLegend; 348814), CD4 PerCP-Cy5.5 (BioLegend; 317427), CD3 AlexaFluor700 (BioLegend; 300424), CD25 FITC (BioLegend; 302604), CD71 PE-Cy7 (BioLegend; 334112) and OX40 AlexaFluor647 (BioLegend; 350017). Flow cytometric data was analysed with FlowJo v10 software (BD). For the mouse FAIM assay, culture supernatants were reserved and frozen on day 7 and frozen for IFN-γ and IL-10 ELISAs (BioLegend 430804 and BioLegend 431414).

### Tetramer Validation and FAIM Assay Staining

Peptide-MHCII monomers were produced by re-folding either the specific GAD65 P10Sol peptide or control peptide comprised of residues 87-101 from the Class II-associated invariant chain peptide (CLIP) with DRB1*04:01 protein (Immunaware). P10Sol (DRB1*04:01)-PE and CLIP₈₇₋₁₀₁(DRB1*04:01)-PE tetramers were produced through the addition of a total of 10 μmol total streptavidin-PE between three 15 min incubations at 4 °C. Tetramer specificity and whether to include protein kinase inhibitor dasatinib in an optimised tetramer staining procedure were assessed using 0.5×10⁶ GAD65 P10 SC3 hybridoma cells in a 96-well plate. Cells were either pre-incubated at 37 °C for 10 mins in media only or media with 5 UM dasatinib before being stained at 37 °C for 1 h in the dark with 15 nM of control or specific tetramer in a stain mix which contained 5 μM dasatinib if the cells received dasatinib pre-treatment. This was followed by α-CD3, α-CD4 and viability stains. P10Sol(DRB1*04:01)-PE tetramer staining of experimental samples from FAIM assays setup with mouse cells from the in vivo tolerance protocols included 5 μM dasatinib as a 10 min pre-incubation step as well as part of the tetramer stain mix for 1 h incubation. Data were acquired on a Fortessa X-20 using DIVA software and analysed with FlowJo v10 software.

### Cell Sorting and TCR Analysis

Human FAIM⁺ CD4 T-cells (CTV^{mid} CD25⁺ CD71⁺) were single-cell sorted using a FACSAria II (BD Biosciences) into 5 μL PBS prepared with DEPC-treated dH₂O on day 7 of the FAIM assay. TCR genes were isolated as described by Eugster and colleagues (Eugster et al., 2013) except TaKaRa Ex Taq was replaced with Phusion Hot Start II DNA polymerase (Thermo F549S). TCR α gene analysis performed using R package sangeranalyseR (Chao et al., 2021).

### Statistics

Data were tested for normal distribution and the appropriate parametric/non-parametric tests were performed using GraphPad Prism v9 (GraphPad) as described in the text. Significance values; * 0.01<p≤0.05, ** 0.001<p≤0.01, *** 0.0001<p≤0.001, **** p≤0.0001.

### EXAMPLES

### FAIM Assay

The Fluorescent dye dilution Activation Induced Marker (FAIM) assay involves a CTV-labelled CD4 cell enriched in vitro cell culture with stimulant(s) of interest and appropriate control(s). The primary readout is flow cytometric assessment of CD4 T-cell CTV dye dilution and cell-surface activation marker expression. Optional paired readouts include 3H-thymidine incorporation and measurements of cytokine secretion. The FAIM assay flow cytometric readout included use of CD25 and OX40, as well-established activation markers, but also CD71 as a new activation induced marker, as expression of CD71 strongly associates with Ki67 and cell proliferation (Lašťovička et al., 2009; Motamedi et al., 2016). Our preliminary data showed that CD71 co-expressed with traditional T-cell activation markers with a potential improvement on the identification and expansion of antigen-reactive human CD4 T-cells (data not shown). In-house development of the FAIM assay as a readout to assess CD4 T-cell tolerance induction used B10.PL mice and a tolerogenic peptide Ac1-9[4Y] derived from Myelin Basic Protein (MBP), a system which had previously been used to demonstrate tolerance induction (Metzler and Wraith, 1993). B10.PL mice which express MHCII H-2^{u} were treated with dose escalation using H-2^{u} specific high-affinity tolerogenic peptide analogue Ac1-9[4Y] and challenge/restimulation with the native peptide MBP Ac1-9[4K] (FIG. 1A). Flow cytometric analysis (gating examples for PBS and MBP Ac1-9[4Y] treated mice shown in FIGS. 1.B and C, respectively) demonstrated that in samples from Ac1-9[4Y] treated B10.PL mice the total blasting cells, dye-diluted proliferating CD4 T-cells and proliferating CD4 T-cells which were co-expressing combinations of activation markers CD25 and CD71 or CD25 and OX40 successfully distinguished tolerance (FIG. 2.A-D). This was further supported by paired 3H-thymidine incorporation and IFN-γ cytokine secretion readouts (FIG. 2E-F). This indicated that CD71 could be used in combination as a new activation induced marker.

A soluble peptide effective at activating glutamate decarboxylase (GAD65) peptide-specific hybridomas was designed (GAD65 P10Sol). P10Sol can bind to steady-state CD11c⁺ antigen presenting cells in an antigen processing independent manner and was selected as a candidate for tolerance induction studies (data not shown). Further validation of the FAIM assay used cells isolated from HLA-DR4 transgenic mice treated with either dose escalation of GAD65 P10Sol or PBS control before challenge with the P10 30-mer in strong adjuvant (FIG. 3A). In vitro re-stimulation used titrations of GAD65 P10 30-mer and GAD65 P10Sol. Flow cytometric analysis (gating strategy for a PBS treated and a P10Sol treated mouse shown in FIGS. 3B and C, respectively) revealed that in P10Sol treated mice, total dye-diluted proliferating CD4 T-cells and those which were co-expressing combinations of activation markers CD25 and CD71 or CD25 and OX40, were either almost absent or dramatically reduced when re-stimulated with P10Sol or P10 30-mer peptides, respectively (FIG. 4A-C). This reduced CD4 T-cell response was supported by paired 3H-thymidine incorporation data, which showed a strong, but not statistically significant, trend towards reduced overall proliferative response in P10Sol treated mice re-stimulated with P10Sol or P10 30-mer (FIG. 4D). To confirm whether proliferating CD4 T-cells which expressed activation markers were antigen-specific, we tetramerised and validated a P10Sol peptide-DR4 MHC class II monomer (P10Sol (DRB1*04:01)-PE) using a P10-specific single-cell (FIG. 5A). P10Sol (DRB1*04:01)-PE tetramer stain was included in samples re-stimulated in vitro with 10 μg/mL of P10Sol or P10 30-mer and showed significantly enriched binding only to proliferating CD4 T-cells (FIG. 5B-C). P10Sol (DRB1*04:01)-PE staining was used to assess P10Sol antigen-specificity within proliferating CD4 T-cells which expressed combinations of activation markers (FIG. 5D). This demonstrated that the activation induced marker combination of CD25/CD71 was significantly enriched for tetramer positive cells compared to CD25 and OX40 (FIG. 5E) and held true if enrichment of activation marker co-expression within total tetramer⁺ responding CD4 T-cells was analysed (FIG. 5F).

### FAIM Assay Validation to Detect Self- or Foreign-Antigen Responses in Human Lymphocytes

Having successfully demonstrated that the FAIM assay could be used to assess whether candidate tolerogenic peptides could induce tolerance in relevant mouse model, it was important to assess whether the FAIM assay could also be used to detect self- or foreign antigen responses in human PBMCs. We first show responses to self-antigen, and so measured paired FAIM⁺ (CD25⁺ CD71⁺ proliferating CD4⁺ T-cell) and ³H-thymidine proliferation responses from PBMCs isolated from 44 adult T1D patients which had been stimulated with T1D relevant self-antigens GAD65 P10 30-mer or P10Sol (FIG. 6A). FAIM⁺ analysis highlighted that 89% (34/38) of T1D patients could respond to GAD65 P10 30-mer, of which 38% (13/34) also responded to P10Sol (FIG. 6A). Importantly, the FAIM⁺ flow cytometric readout detected 16 additional positive responses (10.2% of total outcomes) which were below the positive threshold of the paired ³H-thymidine proliferation readout, whereas only 1 outcome (0.6% of total outcomes) was detected by ³H-thymidine and not FAIM⁺ (FIG. 6A-B). Furthermore, the magnitude of the FAIM⁺ response over background was significantly higher compared to paired ³H-thymidine data, independent of stimulant concentration (FIG. 6C, Wilcoxon matched-pairs sign rank test). These data showcase that the flow cytometric readout of the FAIM assay, which interrogates both proliferation and activation induced responses in CD4 T-cells, is more sensitive than the traditional ³H-thymidine incorporation assay. We also stimulated PBMCs from 22 T1D patients with either foreign or self-antigen. We demonstrate that the FAIM assay can readily detect in vitro CD4⁺ T-cell responses to foreign antigens PPD and KLH (FIG. 7A-B). PBMCs from a healthy donor were stimulated with whole pro-insulin protein before FAIM⁺ (CTV^{mid} CD25⁺ CD71⁺) CD4⁺ T-cell cells were single-cell sorted and the TCR genes were isolated. TCR α gene analysis shows that clonally expanded antigen-responsive, proliferative, activated CD4 T-cells are isolated by the FAIM assay (FIG. 8).

### CONCLUSION

Using our novel FAIM assay and a P10Sol peptide-MHCII tetramer, a higher frequency of antigen-specific proliferating CD4 T cells were identified using only a new combination of activation markers CD25 and CD71, compared to use of only CD25 and OX40. The results of the FAIM assay open the possibility of using CTV dilution, CD25 and CD71 to identify antigen-specific CD4 T cells to any antigen—both foreign (infectious disease) and self (autoimmune or cancer neoantigen). The present study demonstrates that our FAIM assay can identify antigen-responding CD4 T cells stimulated by foreign antigens, PPD and KLH, and autoimmune antigens, MBP; Ac1-9 (Zamvil et al., 1986) and GAD65. Using the FAIM assay it should be possible to identify the dominant epitope of any antigen directly from human PBMCs without the requirement of mouse models. Human PBMCs would be cultured with the antigen of interest with single-cell sorting of proliferating CD25⁺ CD71⁺ CD4 T cells. Downstream single-cell TCR cloning and sequencing, would identify overrepresented TCR sequences, which would be immortalised by transfection into TCR knockout reporter cell lines and re-tested for antigen-reactivity. This would represent an incredibly powerful new workflow with potentially far-reaching implications for antigen-specific immunotherapies beyond T1D, for example, generating antigen-specific regulatory T cells or neo-antigen-specific CD4 T cells for re-infusion into autoimmune or cancer patients.

### REFERENCES

Bowyer et al., 2018. Vaccines (Basel). 2018 Jul. 31; 6(3): 50. doi: 10.3390/vaccines6030050.
Braun et al., 2020. Nature. 2020 November; 587(7833):270-274. doi: 10.1038/s41586-020-2598-9.
Chao et al., 2021. Genome Biol Evol. 2021 Mar. 1; 13(3):evab028. doi: 10.1093/gbe/evab028.
Dan et al., 2016. J Immunol Aug. 1, 2016, 197 (3) 983-993; DOI: https://doi.org/10.4049/jimmunol. 1600318
Eugster et al., 2013. J Immunol Methods. 2013 Dec. 31; 400-401:13-22. doi: 10.1016/j.jim.2013.11.003.
Fugger et al., 1994. Proc Natl Acad Sci USA. 1994 Jun. 21; 91(13):6151-5. doi: 10.1073/pnas.91.13.6151
Havenar-Daughton et al., 2016. J Immunol 2016; 197:994-1002. doi: 10.4049/jimmunol. 1600320
Jiang et al., 2019. J Immunol Methods. 2019 April; 467:48-57. doi: 10.1016/j.jim.2019.02.008
Lašťovička et al., 2009. Cell Immunol. 2009; 256(1-2):79-85. Doi: 10.1016/j.cellimm.2009.01.007
Metzler and Wraith, 1993. Comparative Study Int Immunol. 1993 September; 5(9):1159-65. doi: 10.1093/intimm/5.9.1159.
Motamedi et al., 2016. J Immunol Methods. 2016 October; 437:43-52. Doi: 10.1016/j.jim.2016.08.002
Reiss et al., 2017. PLOS One. 2017 Oct. 24; 12(10):e0186998. DOI: 10.1371/journal.pone.0186998
Zamvil et al., 1986. Nature. 1986 November; 324(6094):258-60. doi: 10.1038/324258a0

1. A method for identifying antigen-specific T cells produced in response to stimulation by an antigen, the method comprising detection of antigen-specific T cell surface expression of CD71.

2. The method according to claim 1 further comprising detection of antigen-specific T cell surface expression of CD25.

3. The method according to claim 1, wherein the method comprises detection of antigen-specific T cell surface expression of CD71 and CD25.

4. The method according to claim 1, wherein the antigen-specific T cells are labelled with a proliferation marker.

5. The method according to claim 4, wherein the proliferation marker comprises a cell-permeant molecule.

6. The method according to claim 1, wherein the method comprises detection of antigen-specific T cell surface expression of CD71 and CD25, and wherein the antigen-specific T cells are labelled with a proliferation marker comprising a cell-permeant molecule.

7. The method according to claim 4, wherein the proliferation marker comprises carboxyfluorescein succinimidyl ester or CellTrace Violet.

8. The method according to claim 4, wherein the method comprises detection of antigen-specific T cell surface expression of CD71 and CD25, and wherein the antigen-specific T cells are labelled with CellTrace Violet.

9. The method according to claim 1, wherein the antigen is a self-antigen or a foreign antigen.

10. (canceled)

11. The method according to claim 1, wherein the antigen is derived from cellular proteins, pancreatic islet protein, peptides, enzyme complexes, ribonucleoprotein complexes, nucleic acids and/or post-translationally modified proteins.

12. (canceled)

13. The method according to claim 11, wherein the antigen is a peptide derived from glutamate decarboxylase 65, tyrosine phosphatase-related islet antigen 2 and/or pro-insulin.

14. The method according to claim 1, wherein the antigen-specific T cells are CD4+ and/or CD8+.

15. The method according to claim 1 further comprising detection of antigen-specific T cell surface expression of CD69, CD40L, OX40, 4-1BB, PD-L1, CD107a, or a combination thereof.

16. The method according to claim 1 further comprising detection of antigen-specific T cell surface expression of CD3, ICOS, or a combination thereof.

17. The method according to claim 1, wherein the detection of antigen-specific T cell comprises the detection of a colorimetric, fluorescent and/or luminescent signal.

18. The method according to claim 1, wherein the detection of the proliferation marker comprises a colorimetric, fluorescent and/or luminescent signal.

19. The method according to claim 1, wherein the detection of the antigen-specific T cells is carried out on T cells cultured for at least four days.

20. The method according to claim 1, wherein the T cells are cultured with a self-antigen or a foreign antigen.

21-27. (canceled)

28. An isolated CD71+ and CD25+ antigen-specific T cell, wherein the T cell is CD4+ and/or CD8+.

29. The isolated antigen-specific T cell according to claim 28, wherein the T cell is CD69+, CD40L+, OX40+, 4-1BB+, PD-L1+, CD107a+, or a combination thereof.

30. The isolated antigen-specific T cell according to claim 29, wherein the T cell is CD3+, ICOS+, or a combination thereof.

31. The isolated antigen-specific T cell according to claim 29, wherein the T cell is proliferative.

32. The isolated antigen-specific T cell according to claim 29, wherein the T cell is labelled with a proliferation marker.

33.-34. (canceled)
